(19) United States
(12) Patent Application Publication
(10) Pub. No.: US 2022/0226231 A1
(43) Pub. Date: Jul. 21, 2022

(54) AGRIMONY EXTRACT AS ANTI-POLLUTION AGENT

(71) Applicant: PIERRE FABRE DERMO-COSMETIQUE, BOULOGNE-BILLANCOURT (FR)

(72) Inventors: Christel FIORINI, TOULOUSE (FR); Philippe JOULIA, VILLENOUVELLE (FR); Patrick BOGDANOWICZ, BALMA (FR)

(73) Assignee: PIERRE FABRE DERMO-COSMETIQUE, BOULOGNE-BILLANCOURT (FR)

(21) Appl. No.: 17/609,161

(22) PCT Filed: May 6, 2020
(86) PCT No.: PCT/EP2020/062606

§ 371 Date: Nov. 5, 2021

(30) Foreign Application Priority Data
May 7, 2019 (FR) FR1904763

(51) Int. Cl. A61K 8/9789; A61Q 19/00; A61Q 17/00

(52) U.S. Cl. CPC A61K 8/9789; A61Q 17/00; A61Q 19/007

(57) ABSTRACT

The present invention relates to the use of an agrimony extract as an anti-pollution agent and to cosmetic compositions containing this extract.

The present invention relates to an agrimony extract and to compositions containing this extract for application in the field of cosmetics and dermatology to combat the harmful effects of pollution on the skin.

### PRIOR ART

The official definition of pollution is “damage to the environment by the introduction into the air, water or soil of materials not naturally present in the environment”. As atmospheric pollution continues to increase, chiefly because of human activity, measures have been put in place to support research focused on reducing or controlling emissions of polluting gases. The consequences of this atmospheric pollution have not only effects on a planetary scale, such as the greenhouse effect and the destruction of the ozone layer, but also at the local level, on the health, well-being, skin and skin appendages of living beings.

The main pollutants have both natural and anthropogenic origins. Mention may be made of the main pollutants that have an impact on the skin: solar radiation, polycyclic aromatic hydrocarbons (PAHs), volatile organic compounds (VOCs), ozone O3, nitrogen and sulfur oxides NOx and SOx, particulate matter, also called suspended particles, and tobacco smoke. In the majority of cases, the effect of these polluting factors on the skin is amplified when combined with other pollutants and/or solar radiation.

Solar radiation is an integral part of the study of the interaction of pollution with the skin since it acts in total synergy therewith, and in most scientific studies it is a question of the tandem of solar radiation and pollution. UV rays, combined with pollutants, generate an increase in free radicals and in lipid peroxidation.

Blue light, emitted by the sun and by certain devices whose lighting is fluorescent or LED (computer, tablet, television, smartphone), would have an influence on photoaging. Its spectrum varies between 380 and 500 nm, so its rays are those among the visible radiation that have the highest energy. Blue light is believed to increase the rate of internal free radicals and melanin production, thus promoting the formation of pigment spots.

PAHs originate from, inter alia, the combustion of wood and organic matter and from exhaust gases. They can be deposited and/or adsorbed on the surface of particulate matter. PAHs can enter the skin via the hair follicles or through the epidermis. They are converted to quinines, cyclic chemical compounds that produce reactive oxygen species. They can also promote acne. One PAH is 2,3,7,8-tetrachlorodibenzo-p-dioxin, a halogenated aromatic hydrocarbon, which is a lipophilic compound capable of activating the aromatic hydrocarbon receptor, which will activate the oxidative cascade (Drakaki et al., 2014).

Volatile organic compounds come from organic solvents in paints, varnishes, lacquers, fuels, tobacco smoke, industrial and automotive emissions. Studies have shown that these compounds induce an increase in cytokines that promote the development of inflammatory and allergic reactions such as atopic dermatitis and psoriasis. The combined action of volatile organic compounds, hydrocarbons, nitrogen oxides, and sunlight can result in the formation of ozone (Drakaki et al., 2014).

Ozone can have a natural origin since it is released in small quantities by the soil and plants. However, it is mainly of anthropogenic origin. Ozone is a strong oxidizing agent capable of modifying the integrity of the skin. In the skin, it induces oxidative stress and lipid peroxidation, respectively decreases the concentration of antioxidant vitamins C and E and increases the concentration of malondialdehyde (MDA). It acts preferentially on the lipids and fatty acids constituting the lipidic matrix of the stratum corneum of the epidermis, in which it will form reactive oxygen species. These processes will upset the epidermal balance and lead to multiple skin disorders via a stimulation of the expression of MMPs which, in turn, will degrade the collagen and elastin fibers of the dermis, accelerating skin aging, tissue sagging and, ultimately, deepening wrinkles (Drakaki et al., 2014).

The oxides involved in skin alterations are mainly nitrogen dioxide, sulfur dioxide and carbon monoxide. Nitrogen dioxide is of anthropogenic origin since it is emitted during combustion. Sulfur dioxide can have the same origin as nitrogen dioxide, but also a natural origin linked to forest fires and volcanic eruptions. Carbon monoxide comes from incomplete combustion. In regions where these oxides are present in high concentrations, an increase in atopic dermatitis is observed in children according to studies conducted in Europe (Drakaki et al., 2014).

Suspended particles are fine solid particles transported by air or water. It is not the particles themselves that are responsible for the deleterious effects but the molecules adsorbed on these particles (benzo (a) pyrene, heavy metals, pesticides, etc.). They can generate oxidative stress on the skin and are thus at least partly responsible for skin aging. They are suspected of inducing the formation of wrinkles and the appearance of pigment spots.

For all these pollutants, the risks and consequences vary according to their concentration, the duration of exposure, their combinations, but above all the predisposition of the subject. All together, they exhibit a synergy of action.

The effects of pollution on the skin are varied, with observations including an acidification of the skin's pH, a decrease in hydration with an increase in transepidermal water loss, an increase in desquamation, a decrease in corneal suppleness, a relaxation of tissues, a modification of surface lipids by the action of free radicals, a decrease in cellular energy metabolism. The repetition of these insults initiates an inflammatory process and predisposes to reactions of intolerance in the longer term.

Moreover, pollution has an effect on the reduction of the skin's protective barrier function by modifying the hydrolipidic film. It will lead to an alteration in cell renewal which will then be slower, the skin will then appear duller, less firm. It is also known that pollution prematurely ages the skin by increasing the production of free radicals that destroy cells and cause the destruction of collagen and elastin fibers.

Faced with this phenomenon, which is growing, a clear sales increase has been observed in hygiene, care and hair products with anti-pollution claims.

For example, patent application WO06111666 discloses a composition containing an extract of Camellia sinensis and an extract of Lapsana communis to combat the consequences of pollution on the skin. Similarly, patent FR 2838342 relates to cosmetic compositions containing an extract of the marine alga Sargassum muticum useful for combating the consequences of oxidative stress induced by reactive oxygen species, ultraviolet radiation and against the effects of pollution.

Kostryco et al. (World Scientific News 89(2017)90-97) describe that an extract of agrimony has anti-inflammatory virtues. This paper certainly mentions anti-inflammatory properties of an agrimony extract, but such an effect is cited among a number of other virtues. For example, Kostryco et al. make an inventory of the phytotherapeutic properties (point 5 page 93) of agrimony extracts and cite anti-HBV activity, anti-influenza activity, antibacterial activity, activity on wound healing, antioxidant activity which they link to anti-inflammatory activity and in particular at the level of the gastrointestinal mucosa, anti-diarrheal activity and anti-cancer activity. This document cites body weakness, epigastric pain, intestinal inflammation, liver disorders, digestive, enteric and gastric disorders but also cholecystitis, flatulence, varicose veins, damaged skin, mouthwash, pharyngitis, respiratory inflammation, asthma, menopausal disorders, protection from cardiovascular disorders, gynecological disorders, problems related to diabetes and urinary tract diseases. This document thus focuses on digestive inflammation but does not mention any dermatological or cosmetological effects.

On the front line, the skin cells, their lipids and proteins, but also their DNA are the targets of pollutants, generating oxidation and inflammation. Dull, dry, reactive and/or sensitive skin, acne, premature and accelerated aging are, among others, the identified consequences of pollution. This pollution leads to an overproduction of free radicals in the skin that disrupt the skin's balance and accelerate aging. Just like UV rays or smoking, pollution is therefore an additional risk factor.

The skin, in particular the upper layer of the epidermis, constitutes a barrier against external insults such as chemical, mechanical or infectious insults and against environmental factors (climate, ultraviolet rays, pollutants and/or xenobiotics). This property, known as the barrier function, is chiefly provided by the most superficial layer of the epidermis, namely the stratum corneum.

One of the functions of the stratum corneum is to absorb and retain water from the epidermis, and any alteration in its structure and/or function can lead to changes in skin hydration, particularly increased water loss. Excessive dry skin can, in a vicious circle, lead to an alteration in the skin's barrier function, and increased damage due to environmental stress.

A skin barrier disorder can be generally defined as a condition in which the skin, or more particularly the epidermis and in particular the stratum corneum, does not exhibit normal barrier function. This deterioration of the skin may be simply a minor cosmetic alteration and/or a sensation of skin discomfort or dryness.

Thus, despite the few currently available options, consumers need new compounds or compositions useful for combating the harmful effects of pollution on skin, skin tone, skin texture and firmness, as well as on hair.

### SUMMARY OF THE INVENTION

Surprisingly, the inventors discovered that an extract of agrimony, in particular of its aerial parts, more particularly the flowering tops, protected the skin and the skin appendages as well as the keratinous material against the harmful effects of pollution (Example 2).

The invention thus relates to a novel use of an agrimony extract as anti-pollution cosmetic agent, and to the use of anti-pollution cosmetic compositions containing this extract.

In particular, the invention is directed to a non-therapeutic cosmetic use, as an agent for preventing or treating the effects of atmospheric pollutants on the skin and/or skin appendages, of an agrimony extract, in particular an extract of the aerial parts of agrimony, more particularly the flowering tops, said extract being in a cosmetic composition further comprising a cosmetically acceptable excipient.

The invention thus relates to a non-therapeutic cosmetic use of an effective amount of an agrimony extract, in particular an extract of the aerial parts of agrimony, more particularly the flowering tops, to prevent or treat the effects of atmospheric pollutants on the skin and/or skin appendages.

The effects of atmospheric pollutants on the skin can be selected from sagging skin, gray complexion, clogged pores, loss of radiance.

The effects of atmospheric pollutants on the skin appendages can be selected from dull hair, dry hair, brittle hair and oily hair.

The invention also relates to the use of an agrimony extract, in particular an extract of the aerial parts of agrimony, more particularly the flowering tops, in a cosmetic composition, for preventing or treating the effects of atmospheric pollutants on the skin and/or skin appendages.

According to another aspect, the invention relates to a composition or extract according to the invention for use in preventing or treating the effects of atmospheric pollutants on the skin and/or skin appendages;

Also, according to one aspect of the invention, the invention is directed to a method for preventing or treating the effects of atmospheric pollutants on the skin and/or skin appendages comprising the topical application of a composition according to the invention.

Also, according to one aspect of the invention, the invention is directed to a method for preventing or treating the effects of atmospheric pollutants on the skin and/or skin appendages comprising the oral administration of a composition according to the invention.

Finally, the invention also relates to the use of an extract according to the invention for the manufacture of a cosmetological or dermatological composition intended for the treatment or prevention of the effects of atmospheric pollutants on the skin and/or skin appendages. According to a particular case, said cosmetological or dermatological composition is a composition suitable for topical application. According to another case said cosmetological or dermatological composition is a composition suitable for oral administration.

In particular, the skin and/or skin appendages is skin or skin appendages exposed to an urban environment. Indeed, an urban environment is particularly loaded with polluting agents and skin or skin appendages thus exposed require a treatment such as that of the invention.

The invention is therefore also directed to a cosmetic method for preventing or treating the effects of atmospheric pollutants on the skin and/or skin appendages of a person, in particular the skin of the face, said method comprising the topical application of a cosmetic composition comprising an agrimony extract, in particular an extract of the aerial parts of agrimony.

In particular, the person to whom the cosmetic composition is applied is a person whose skin and/or skin appendages, in particular the skin of the face, is/are exposed to an urban environment.

It is also a subject matter of the present invention to provide a use of an extract of agrimony according to the invention, in particular of its aerial parts, more particularly the flowering tops, to counter damage to the human skin barrier. In particular it is damage that can occur due to the effect of environmental insults, in particular pollution.

In particular, the inventors of the present invention have discovered, inter alia, that an extract of agrimony according to the invention, in particular of its aerial parts, more particularly the flowering tops, has a significant protective effect on human skin cells, in particular the barrier layer, against several common atmospheric pollutants.

In the context of the present invention, an extract of agrimony according to the invention, in particular of its aerial parts, more particularly the flowering tops, can be used for application to healthy skin which is or may be subject to the influence of environmental stresses such as climatic imbalances or pollutants and which may therefore cause skin discomfort or dryness.

In other specific cases, the extract of agrimony according to the invention, in particular of its aerial parts, more particularly the flowering tops, can be applied to the skin when it already shows clinical signs of a deficiency of the skin barrier.

One aspect of the present invention relates to the non-therapeutic or cosmetic use of the extract of agrimony according to the invention, in particular of its aerial parts, more particularly the flowering tops, to combat damage to the human skin barrier caused by environmental insults.

According to another aspect, the present invention relates to an extract of agrimony according to the invention, in particular of its aerial parts, more particularly the flowering tops, for use in the treatment and/or prevention of human skin barrier disorders and associated signs.

According to another aspect, the present invention relates to the use of extract of agrimony according to the invention, in particular of its aerial parts, more particularly the flowering tops, for the preparation of a composition intended to combat damage to the human skin barrier caused by environmental insults and/or to prevent and/or treat skin barrier disorders and the associated signs.

According to another aspect, the present invention relates to a method for countering damage to the human skin barrier caused by environmental insults, the method comprising administering to the subject in need thereof an effective amount of an extract of agrimony according to the invention, in particular of its aerial parts, more particularly the flowering tops.

According to another aspect, the present invention relates to a method for the treatment and/or prevention of human skin barrier disorders and associated signs, said method comprising administering to the subject in need thereof an effective amount of an extract of agrimony according to the invention, in particular of its aerial parts, more particularly the flowering tops.

Another aspect of the invention relates to a cosmetic or dermocosmetic composition intended to combat damage to the human skin barrier caused by environmental insults, said composition comprising an effective amount of an extract of agrimony according to the invention, in particular of its aerial parts, more particularly the flowering tops, in a cosmetically or dermocosmetically acceptable medium.

Advantageously, the agrimony extract according to the invention, in particular its aerial parts, more particularly the flowering tops, offers protection against environmental insults, in particular environmental insults not related to UV, whether from the sun or from artificial sources (including UVA and UVB rays), which can affect the human skin barrier function and cause skin dryness.

These factors include exposure to atmospheric pollution, particularly chemical atmospheric pollutants (for example smoke (such as tobacco smoke), heavy metals (such as arsenic, cadmium, chromium), polycyclic aromatic hydrocarbons (PAHs), volatile organic compounds (VOCs), oxides, particulate matter (PM) and ozone (O3)), and climatic conditions other than UV radiation, such as osmotic shock, low humidity, environmental thermal stress, etc.

The extract of agrimony according to the invention, in particular of its aerial parts, more particularly the flowering tops, or a composition thereof according to the invention may in general be intended to improve the general condition of the human skin, and in particular to counter environmental insults and to prevent and/or treat disorders of the human skin barrier and the associated signs.

In a particular embodiment, an extract of agrimony according to the invention, in particular of its aerial parts, more particularly the flowering tops, or a composition thereof is intended to prevent, treat and/or reduce discomforts, unpleasant phenomena or skin dryness of human skin subjected to the influence of environmental insults such as pollution, in particular non-UV related environmental insults.

In another particular embodiment, an extract of agrimony according to the invention, in particular of its aerial parts, more particularly the flowering tops, or a composition thereof is intended to prevent, treat and/or improve a human skin barrier disorder and associated signs, the skin barrier disorder being selected from dry skin.

Human skin barrier disorders and associated signs comprise all changes in the external appearance of the skin due to the action of environmental insults, in particular environmental insults not related to UV, and to dehydration of the skin (especially the epidermis), such as rough and scaly appearance, and decreased suppleness, but also induced skin sensations or discomforts such as itching and/or tightness.

An extract of agrimony according to the invention, in particular of its aerial parts, more particularly the flowering tops, or a composition thereof according to the invention may in particular be intended to prevent, treat and/or improve skin dryness and/or skin disorders associated with a state of skin dryness, in particular the dryness of an epidermis, and more particularly a lack of hydration of the stratum corneum.

### DETAILED DESCRIPTION

According to various authors, the genus Agrimonia comprises 12 to 15 species, among which the eupatorial agrimony (Agrimonia eupatoria L.) which is a species of nitrophilous, rhizomatous perennial herbaceous plant of the Rosaceae family, common throughout Europe.

It is a plant with long, spindly spikes, with small yellow flowers, with stems of 0.30 to 0.60 m. Its thick stump with twiggy roots emits a simple or little branched, hairy, reddish erect stem carrying large leaves divided into unequal segments. The alternate leaves are composed of large, oval, toothed leaflets alternating with much smaller ones. The leaves are hairy-grayish underneath and without scent glands. The small yellow flowers are numerous and grouped in long terminal clusters that bloom from June to August; they each include a calyx whose tube is marked with 10 grooves and bears several rows of small, soft, hooked spines at the top, and a corolla with 5 oval, spreading petals. The fruits are strongly furrowed along their entire length and their hooked spines are not curled. The fruit consists of 1 or 2 achenes enclosed in the bristly and persistent calyx whose throat is completely closed. It is a perennial plant, common in cool places, woods or uncultivated land.

This species is not found on mountains at great altitudes, common in general throughout France, it is found throughout Europe except in the northern part of the Scandinavian peninsula and in the Arctic regions, it is also found in the northern and western regions of Asia, North Africa, the Canary Islands. The plant is harvested throughout the summer, gathered in bunches and dried in the shade on racks or ropes.

Currently, agrimony is used as a mild astringent internally and externally, used in pharyngitis, gastroenteritis and intestinal disorders. In traditional medicine, agrimony is used in cholecystopathy. The flowering tops are traditionally used in the symptomatic treatment of light diarrhea (oral route), subjective manifestations of venous insufficiency (heavy legs) and in hemorrhoidal symptomatology (oral and local route), mouthwashes for oral hygiene.

Agrimony contains phenolic compounds, volatile compounds and triterpenic compounds. In the leaves there are condensed tannins, accompanied by small amounts of ellagitannins and traces of gallotannins in the stems, silica, flavonoids (luteolin, apigenin and kaempferol heterosides). The plant contains dimeric (B-1, B-3, B-6, B-7), trimeric (C-1, C-2, and others) procyanidols.

Fractionation of the aqueous extract with ethyl acetate identified several phenolic compounds:
agrimoniin,
p-coumaric acid, ellagic acid, 3,3′-di-O-methyl ellagic acid, quinic acid,
catechin
caffeoyl hexoside
apigenin 8-C-glucoside (vitexin)
apigenin
apigenin 6-C-glucoside (isovitexin)
apigenin 7-O-glucoside (apigetrin)
apigenin 7-O-glucuronide
luteolin
luteolin 7-O-glucoside (cynaroside)
luteolin 7-O-glucuronide
luteolin acetyl hexoside
quercetin O-galloyl-hexoside
quercetin 3-O-glucoside (isoquercetin)
quercetin O-malonylhexoside
quercetin 3-O-rhamnoside
quercetin 7-O-rhamnoside
quercetin-acetylglucoside
kaempferol
kaempferol 3-O-glucoside (astragalin)
kaempferol O-malonylhexoside
kaempferol O-rhamnoside
kaempferol O-p-coumaroylglucoside (tiliroside)
kaempferol O-acetylhexosyl-O-rhamnoside
kaempferol-7-O-alpha-L-rhamnoside
rutin

By entrainment with water vapors from the root and leaves, several volatile compounds have been identified in this species. In a study (Feng et al., 2013, JAMC, ID246986, 9 pages), 87 volatile compounds were separated and identified, they represent in total 87% of the total detected constituents. From the quantitative point of view, the representative compounds are the following:
cedrol
α-pinene
linalool
1-(2-furyl)-1-hexanone
α-terpineol
bornyl acetate
eucalyptol
camphene
α-cedrene
patchoulol

Finally, triterpene compounds have been identified: ursolic acid, 19 a-hydroxy-ursolic acid, oleanolic acid, tormic acid.

### Extract According to the Invention

“Agrimony extract” is understood to mean the product of extraction of all or part of the Agrimonia eupatoria plant, preferably its flowering tops.

“Extraction product” is understood to mean the product obtained after extraction of the aerial parts of the Agrimonia eupatoria plant, and in particular the flowering tops of Agrimonia eupatoria, with a solvent, called extraction solvent, (i.e., a liquid solution in the extraction solvent) possibly in a concentrated or dry form after partial or total evaporation of the extraction solvent. It may therefore be a dry extract.

In the sense of the present invention, “dry extract” is understood to mean an extract free of extraction solvent or containing only a trace amount thereof. Such a dry extract thus contains only material from Agrimonia eupatoria, in particular from its flowering tops. It may further contain insignificant traces of extraction solvent. It may be associated with a drying medium such as maltodextrins, starches, silicas, etc.

In the context of the present invention, the Agrimonia eupatoria extract is advantageously obtained from the flowering tops of the plant.

The parts of the agrimony plant, in particular its flowering tops, may be fresh, frozen or dry, whole; cut or ground and then subjected to an extraction step.

A process for preparing an extract according to the invention comprises a step of extracting Agrimonia eupatoria, in particular its flowering tops, with an extraction solvent. The extraction may be carried out at room temperature (for example between 15 and 30° C., in particular between 20 and 25° C.) or hot (between 40 and 250° C. and/or at atmospheric pressure or high pressure.

The extraction solvent may be a solvent selected from:
water
C1 to C5 alcohols, for example ethanol, methanol, isopropanol
C3 to C5 glycols, for example propylene glycol, butylene glycol, pentylene glycol
ketones including acetone and methyl ethyl acetone
hexane, heptane
ethyl acetate
CO2,
mixtures thereof.

Advantageously, the extraction solvent is a hydroalcoholic solvent, i.e., a C1 to C5 alcohol/water mixture, such as an ethanol/water mixture.

More particularly when the solvent is hydroalcoholic, it may be a C1-05 alcohol/water mixture in an alcohol/water volume ratio comprised between 10/90 and 90/10, preferentially between 20/80 and 40/60. Preferably, the extraction solvent is an ethanol/water mixture in a volume ratio of the order of 10/90 to 90/10, more particularly between 20/80 and 40/60. Even more particularly, the extraction solvent is an ethanol/water mixture in a volume ratio of 30/70.

The extraction can be carried out by different technologies, in particular so-called “green” technologies, such as at atmospheric pressure (possibly using extraction assistance means such as ultrasound, microwaves, leaching, maceration, decoction, etc.) or at high pressures, with extraction solvents in the super- or sub-critical state such as CO2 with or without a co-solvent such as ethanol or water, and/or at temperatures comprised between 20° C. and 250° C.

For the extraction, the ratio of solvent to agrimony parts may be comprised, by mass (g)/volume (mL), between ⅕ and 1/20, particularly between ⅛ and 1/12.

The agrimony extract, in particular an extract of its flowering tops, will advantageously consist of a phenolic fraction.

“Phenolic fraction” is understood to mean a fraction of the extract containing compounds with one or more phenol groups such as flavonoids and tannins.

According to a first aspect, the invention relates to the use of an extract of the aerial parts of agrimony as an anti-pollution cosmetic agent.

“Anti-pollution cosmetic agent” is understood to mean an ingredient of a cosmetic composition having the effect of preventing or treating the effects of atmospheric pollutants on the skin and/or skin appendages.

According to a second aspect, the present invention relates to a dermatological or cosmetic composition comprising at least one extract of the aerial parts of agrimony with at least one dermatologically or cosmetically acceptable excipient for use in preventing or treating the effects of atmospheric pollutants on the skin and/or skin appendages.

“Skin appendages” is understood to mean the hair of the head or body, the nails or any other keratinous material. Preferably, it is the hair.

Advantageously, the extract comprised in the dermatological or cosmetic composition is as described above.

According to a particular embodiment of the invention, the dermatological or cosmetic composition comprising at least one extract of the aerial parts of agrimony as described above and at least one dermatologically or cosmetically acceptable excipient comprises from 0.01 to 10% by weight, preferably from 0.05 to 5% by weight, in a preferred manner from 0.1 to 3% by weight, still more preferably from 0.5 to 2% by weight of extract of the aerial parts of agrimony, by weight of dry extract based on the total weight of the composition.

The invention is preferably directed to a dermatological or cosmetic composition according to the invention which is in a form suitable for topical application, in particular to the skin, scalp and/or hair.

The cosmetic or dermatological composition according to the invention can thus be in the forms usually known for topical administration, i.e., in particular, lotions, shampoos, balms, foams, gels, dispersions, emulsions, sprays, serums, masks or creams, with excipients which can, in particular, allow penetration in order to improve the properties and the accessibility of the active principle.

Advantageously, it will be a milk, a cream or a balm.

The invention is thus directed to dermatological or cosmetic compositions according to one of the embodiments of the present invention, characterized in that they are in a form suitable for topical application.

These compositions generally contain, in addition to an agrimony extract according to the invention, in particular an extract of the aerial parts of agrimony, a physiologically acceptable medium, in particular one or more cosmetically or dermatologically acceptable excipients, generally based on water or solvent, for example alcohols, ethers or glycols. They may thus further contain surfactants, complexants, preservatives, stabilizers, emulsifiers, thickeners, gelling agents, humectants, emollients, trace elements, essential oils, perfumes, dyes, mattifying agents, chemical or mineral filters, moisturizing agents or geothermal waters, anti-UV agents or sun filters, for example.

The cosmetic or dermatological composition according to the invention may further contain at least one other anti-pollution compound selected from sunscreens, polysaccharides and pearlescent agents.

According to an alternative embodiment, the present invention also relates to a composition comprising an extract of the aerial parts of agrimony as described, in a form suitable for oral administration.

The invention is thus directed to the use of an anti-pollutant cosmetic composition according to the invention, characterized in that the composition is intended for oral administration.

In particular, the invention relates to the use of an anti-pollutant cosmetic composition comprising at least one extract of the aerial parts of agrimony, with at least one pharmaceutically acceptable excipient, for preventing or treating the effects of atmospheric pollutants on the skin and/or skin appendages, characterized in that the composition is intended for oral administration.

In particular, it may be a hard capsule, soft capsule, tablet or drinkable powder containing the extract of the aerial parts of agrimony as described herein in combination with usual pharmaceutically acceptable excipients such as bulking agents, stabilizers and dyes traditionally used in the galenic formulation of oral pharmaceutical forms. Such oral forms may comprise 0.01 to 10% by weight, preferably 0.05 to 5% by weight, in a preferred manner 0.1 to 3% by weight, more preferably 0.5 to 2% by weight of extract of the aerial parts of agrimony, by weight of dry extract based on the total weight of the composition.

The cosmetological or dermatological composition, in a form suitable for topical application or oral administration, according to the invention, may further contain at least one other plant extract.

The following examples illustrate the invention without limiting its scope.

### EXAMPLE 1

### Preparation of the Extract

Introduce in a reactor 100 g of ground aerial parts with 1 liter of 30% (v/v) ethanol,
Extract at room temperature for 2 hours with shaking (the extraction is performed twice),
Filter at room temperature,
Collect the filtrates,
Focus,
Sterilize for 20 minutes at 120° C. if necessary,
Dry out,
The solution obtained constitutes the hydroalcoholic extract of the aerial parts of agrimony.

The yield is 12 g/100 g of flowering tops.

### EXAMPLE 2

### Anti-Pollution Activity

The objective of this study is to evaluate the antioxidant, detoxifying and ozone-protective activity of an agrimony extract, following exposure to ozone in a reconstructed human epithelium (RHE) mimicking the effect of pollution on the skin. Lipids playing a role in epidermal barrier function, such as free fatty acids, cholesterol, triglycerides and ceramides, can be impacted under stress conditions with increased oxidative lipid damage. Direct measurements of lipid peroxidation can be carried out by measuring malondialdehyde (MDA) by gas chromatography techniques combined with mass spectrometry. This compound is naturally present in tissues, where it is a manifestation of oxidative stress. It is derived in particular from the action of reactive oxygen derivatives on polyunsaturated fatty acids.

Several conditions are realized in this study:
Negative control: non-stressed RHE;
Positive control: ozone-stressed RHE;
Test with an agrimony extract: ozone-stressed RHE is in contact with the extract (n=3), this test is performed with 3 different concentrations (1, 3 and 10 μg/mL);
Test with a reference compound: ozone-stressed RHE is in contact with the reference compound (Trolox).

3,4-Dihydro-6-hydroxy-2,5,7,8-tetramethyl-2H-1-benzopyran-2-carboxylic acid, or Trolox, is a hydrophilic analogue of vitamin E. It is an antioxidant, which is used to limit damage due to oxidative stress. It is an antioxidant, which is used to limit damage due to oxidative stress.

The agrimony extract is solubilized in water/DMSO, (80/20, v/v). The process is based on lipid oxidation from ozonolysis. Ozone is produced at 0.2 L/min by a mixture of O2 and O3 (99/1% (v/v)) in the medium. The production of 1% ozone produced by this process is equivalent to 4 ppm of ozone exposure. The ozonolysis reaction induces various products called primary or secondary products according to the Criegee rearrangement.

RHEs are constructed from normal human primary keratinocytes obtained from subjects who have undergone abdominal dermolipectomy. After 8 days of exposure to air, the medium is changed and the test compound is added at the desired concentration. On day 9, the RHEs are placed in a chamber with a new medium. The reaction medium is cooled to 5° C. and under bubbled air for 1 minute. The air production is 0.2 L/min at approximately 99% O2 and 1% O3 (v/v). Then the RHE are introduced into a plate and a new medium (without active agent) is added. On day 10, the RHE and media are recovered.

A protection index is calculated from the stress and control conditions.

PI(%)=(stress condition−(stress+active agent condition))/(stress condition−control condition)*100.

The statistical evaluation is based on a one-way ANOVA followed by the Bonferroni multiple comparison test.

### Results

MDA production (ng/mg protein) is shown for each condition tested averaged for the 3 donors in Table 2 below

TABLE 1

| | | | |
|---|---|---|---|
| Concentration | Condition/product | [MDA] | PI (%) |

| | | | | |
|---|---|---|---|---|
| | | Negative control | 18.28 | — |
| | | Positive control, O3 | 53.58 | — |
| 1 | mg/mL | Trolox | 40.32 | 37.6 |
| 1 | μg/mL | Agrimony extract | 48.27 | 15.0 |
| 3 | μg/mL | | 39.80 | 39.1 |
| 10 | μg/mL | | 34.86 | 53.0 |

This test is particularly robust and reliable. The values of MDA concentrations in the control group are relatively homogeneous with a mean value of 18.3±0.7 ng/mg protein. For stress conditions, with ozone exposure, MDA production is strongly increased, this increase is statistically significant (p<0.001) compared with the control group (without ozone). The variability of the responses is also low, the mean value reaches 53.6±3.2 ng/mg protein.

Trolox tested at 1 mg/mL significantly (p=0.0062) reduces MDA production, this expected result validates this test.

The agrimony extract significantly, and in a concentration-dependent manner, reduces MDA production. At 3 μg/mL, this reduction is already significant (p=0.0022), at 10 μg/mL the protection index reaches more than 50% (p=0.0001) and thus largely exceeds that found with the reference product (trolox: 38%).

The inventors show that an agrimony extract induces a protective effect against lipid peroxidation due to ozone, demonstrating an anti-pollution effect.

1. Cosmetic method for preventing or treating the effects of atmospheric pollutants on the skin and/or skin appendages by using an extract of the aerial parts of agrimony Agrimonia eupatoria as an anti-pollution active agent.

2. Method according to claim 1, wherein said extract is obtained from the flowering tops of the plant.

3. Method according to claim 1, wherein said extract is hydrophilic.

4. Method according to claim 1, wherein the use is by topical application.

5. Method for preventing or treating the effects of atmospheric pollutants on the skin and/or skin appendages by using an anti-pollutant cosmetic composition comprising at least one extract of the aerial parts of agrimony Agrimonia eupatoria, with at least one cosmetically acceptable excipient, wherein said extract is obtained from the flowering tops of the plant.

6. Method according to claim 5, wherein the anti-pollutant cosmetic composition comprises 0.05 to 5% of agrimony extract by weight of dry extract based on the total weight of the composition.

7. Method according to claim 5, wherein the composition is intended for topical application.

8. Method for preventing or treating the effects of atmospheric pollutants on the skin and/or skin appendages by using an anti-pollutant cosmetic composition comprising at least one extract of the aerial parts of agrimony Agrimonia eupatoria, with at least one pharmaceutically acceptable excipient, wherein the composition is intended for oral administration.

9. A method for the treatment and/or prevention of human skin barrier disorders and associated signs, said method comprising administering to the subject in need thereof an effective amount of an agrimony extract as defined in claim 1.

10. The method as claimed in claim 9, wherein the human skin barrier disorders and associated signs comprise all changes in the external appearance of the skin due to the action of environmental insults and to dehydration of the skin.

11. The method as claimed in claim 9, wherein the human skin barrier disorders and associated signs comprise rough and scaly appearance, decreased suppleness, skin sensations or discomforts.

12. The method as claimed in claim 9, wherein the human skin barrier disorders and associated signs comprise itching and/or tightness.

13. Method according to claim 1, wherein said extract is hydroalcoholic.
